(19) United States
(12) Patent Application Publication
(10) Pub. No.: US 2024/0241111 A1
(43) Pub. Date: Jul. 18, 2024

(54) DIAGNOSING AND TREATING CRITICALLY ILL SUBJECTS

(71) Applicant: RIGSHOSPITALET, København Ø (DK)

(72) Inventors: Pär Ingemar JOHANSSON, Lund (SE); Hanne Hee HENRIKSEN, København N (DK)

(21) Appl. No.: 18/558,566

(22) PCT Filed: May 5, 2022
(86) PCT No.: PCT/EP2022/062100

§ 371 Date: Nov. 2, 2023

(30) Foreign Application Priority Data
May 6, 2021 (EP) 21172378.8

(51) Int. Cl. G01N 33/53; G01N 33/74

(52) U.S. Cl. CPC G01N 33/5308; G01N 33/74; G01N 2800/52

(57) ABSTRACT

The present invention relates to a medicament for treatment of critically ill subjects with elevated levels of succinic acid, and a method of identifying patients likely to benefit from treatment with a medicament capable of inhibiting catecholamine release and/or blocking adrenergic receptors.

### TECHNICAL FIELD

The present invention relates to a medicament for treatment of critically ill subjects, such as subjects suffering from trauma, sepsis and out-of-hospital-cardiac arrest (OHCA) patients, and a method of identifying the patients most likely to benefit from such a treatment.

### BACKGROUND

More than 800.000 trauma patients and 400.000 sepsis patients die annually in the EU and approximately 40-50% of these deaths occur within 72 hours from injury and/or debut of systemic infection (Krug et al., 2002, Rudd et al., 2010). Likewise, 50-89% of OHCA patients die in the hospital with a global incidence of 55 per 100,000 person-years (Berdowski et al., 2010).

The management of trauma patients differ depending on the cause of the trauma. Firstly, a primary survey is performed in order to evaluate the patient's airways, breathing, circulation, and disability in order to focus on stabilising the patient's status. The continued management of the patient depends on the outcome of the survey as well as the additional tests and examinations that are performed. Similarly, the management of sepsis patients differ depending on the type—and severity of the systemic infection. In general, a patient suffering from sepsis will be treated with intravenous fluids as soon as possible. Treatment with a broad spectrum antibiotic is initiated early on as well, but this might be switched to a different type of antibiotic targeting a specific type of bacteria depending on the results from the microbiologic.

As a critically ill patient may suffer from very low blood pressure, which remains low even after intravenous fluids, the patient might receive vasopressors, which constricts the blood vessels and increases the blood pressure. It is also important to apply measures, which help stabilize breathing and heart function.

Succinic acid is a dicarboxylic acid that is generated in mitochondria via the citric acid cycle. Its function as a biomarker has been suggested in several areas, such as detection of pancreatic cancer or an autism spectrum disorder, or to predict preterm delivery (WO 2015/064594, CA 2 940 906, WO 2017/192668). Succinic acid has also been suggested as a predictor of mortality in critically injured patients (D'Alessandro et al., 2017).

Beta-blockers, or beta-adrenergic receptor antagonists, are a class of medications that block the receptor site for endogenous catecholamines, e.g. adrenaline and noradrenaline, on beta-adrenergic receptors. Beta-adrenergic receptors are a part of the sympathetic nervous system and, when activated, mediates a fight-or-flight and stress response. Beta-blockers are mainly used to manage abnormal heart rhythms, arterial hypertension and as a protective treatment after a myocardial infarction. The use of beta-blocker therapy in critically ill subjects is traditionally regarded as a contra-indication.

Due to the high mortality of critically ill subjects and limited treatment options for such patients, there is a need in the art for new studies and inventions that could result in improved diagnosis and treatment of these patients. An improved stratification of critically ill subjects by identifying groups of patients that would benefit from certain treatments, has the potential to decrease the unprecedented high early mortality risk of these patients. This would save hundreds of thousands of lives worldwide annually as well as substantial reduction in societal costs.

### SUMMARY

The inventors of the present invention have developed a method of identifying a novel group of critically ill subjects who have a significantly higher risk of early death, i.e. death within 72 hours from injury, compared to other critically ill subjects. The term “Toxic Catecholamine Syndrome” is used in reference to critically ill subjects having toxic hyperactivation of the sympathetic nervous system.

The inventors have found that this group of patients can be specifically identified and diagnosed by elevated levels of succinic acid. The inventors have further found that this group of patients will likely benefit from treatment with a medicament that inhibits catecholamine release and/or blocks adrenergic receptor blockers, such as beta-blockers—a treatment which is usually considered a contra-indication in the acute management of critically ill subjects, such as critically ill trauma, sepsis and OHCA patients.

In one aspect of the present invention, the invention relates to a medicament which inhibits catecholamine release and/or blocks adrenergic receptors for use in the treatment of a subject suffering from Toxic Catecholamine Syndrome.

In another aspect of the invention, the invention relates to a method of identifying a subject who is likely to benefit from treatment with a medicament which inhibits catecholamine release and/or blocks adrenergic receptors, wherein said method comprises the steps:
(a) providing a sample obtained from said subject;
(b) determining the concentration of succinic acid in said sample;
   wherein a subject having an elevated succinic acid level compared to a reference level is likely to benefit from treatment with a medicament capable of inhibiting catecholamine release and/or blocking adrenergic receptors.

In one aspect of the present invention, said medicament is used in the manufacture of a medicament for the treatment of a subject suffering from Toxic Catecholamine Syndrome.

### DESCRIPTION OF DRAWINGS

FIG. 1. Succinic acid functions as a predictor for 72-hour mortality in trauma patients. The figure shows the Receiver operator characteristic (ROC) analysis and identification of an optimal cut-off for the plasma biomarker Succinic acid for diagnosing early death (within 72 hours from injury) in 86 trauma patients. The optimal cut-off was defined as the Youden index maximizing the sum of sensitivity and specificity. This ROC curve displays the results of a Succinic cut-off threshold of 15 μmol/L, which exhibits a sensitivity of 0.889, a specificity of 0.662 and a ROC AUC of 0.771 (95% confidence interval 0.596, 0.945).

FIG. 2. Succinic acid functions as a predictor for 72-hour mortality in sepsis patients. The figure shows the Receiver operator characteristic (ROC) analysis and identification of an optimal cut-off for the plasma biomarker Succinic acid for diagnosing early death (within 72 hours from injury) in 179 sepsis patients. The optimal cut-off was defined as the Youden index maximizing the sum of sensitivity and specificity. This ROC curve displays the results of a Succinic cut-off threshold of 10 μmol/L, which exhibits a sensitivity of 0.833, a specificity of 0.568 and a ROC AUC of 0.790 (95% confidence interval 0.692, 0.888).

FIG. 3. Succinic acid functions as a predictor for 72-hour mortality in OHCA patients. The figure shows the Receiver operator characteristic (ROC) analysis and identification of an optimal cut-off for the plasma biomarker Succinic acid for diagnosing early death (within 72 hours from injury) in 98 OHCA patients. The optimal cut-off was defined as the Youden index maximizing the sum of sensitivity and specificity. This ROC curve displays the results of a Succinic acid cut-off threshold of 7 μmol/L, which exhibits a sensitivity=0.750, a specificity=0.709, and a ROC AUC=0.760 (95% confidence interval 0.632, 0.888).

FIG. 4. Trauma patients with Toxic Catecholamine Syndrome have higher levels of Succinic acid compared to trauma patients without Toxic Catecholamine Syndrome. The figure displays a box plot showing the levels of succinic acid (μmol/L) for 86 trauma patients with and without Toxic Catecholamine Syndrome, which shows that trauma patients with Toxic Catecholamine Syndrome on average exhibits 3.9 times higher levels of succinic acid compared to individuals of the cohort without Toxic Catecholamine Syndrome.

FIG. 5. Sepsis patients with Toxic Catecholamine Syndrome have higher levels of Succinic acid compared to sepsis patients without Toxic Catecholamine Syndrome. The figure displays a box plot showing the levels of succinic acid (μmol/L) for 179 sepsis patients with and without Toxic Catecholamine Syndrome, which shows that sepsis patients with Toxic Catecholamine Syndrome on average exhibits 2.3 times higher levels of succinic acid compared to individuals of the cohort without Toxic Catecholamine Syndrome.

FIG. 6. OHCA patients with Toxic Catecholamine Syndrome have higher levels of Succinic acid compared to OHCA patients without Toxic Catecholamine Syndrome. The figure displays a box plot showing the levels of Succinic acid (μmol/L) for 98 OHCA patients with and without Toxic Catecholamine Syndrome, which shows that OHCA patients with Toxic Catecholamine Syndrome on average exhibits 3.7 times higher levels of Succinic acid compared to individuals of the cohort without Toxic Catecholamine Syndrome.

### DETAILED DESCRIPTION

The inventors of the present invention have studied different cohorts of critically ill subjects that died within 72 hours after arrival at the trauma centre.

A trauma patient is a subject who has suffered a serious or life-threatening injury as a result of an event e.g. a car accident, gunshot wound or fall. Traumatic injuries can range from minor isolated wounds to complex injuries involving multiple organ systems. Examples of traumatic injuries are head, neck and spine trauma, chest trauma and abdominal and pelvic trauma.

As approximately 40-50% of all trauma deaths occur within 72 hours from injury, this represents a critical group of patients. A further finding was the identification of a metabolic biomarker; succinic acid, that specifically could identify these trauma patients with increased risk of (early) death. A succinic acid cut-off level of 15 μmol/L provided a sensitivity=0.889, a specificity=0.662 and a ROC AUC=0.771 (95% confidence interval 0.596, 0.945).

A sepsis patient is a subject who suffers a life-threatening organ dysfunction caused by a dysregulated host response to infection and septic shock is defined as persisting hypotension requiring vasopressors to maintain MAP [mean arterial pressure]>65 mmHg and having a serum lactate level >2 mmol/L (18 mg/dL) despite adequate volume resuscitation. Examples of organ dysfunctions are pulmonary, liver, cardiac, renal, and haematological.

As approximately 40% of all sepsis deaths occur within 72 hours from hospital admission, this represents a critical group of patients. A further finding was that also in this group of patients the metabolic biomarker; succinic acid, could specifically identify these sepsis patients with increased risk of (early) death. A succinic acid cut-off level of 10 μmol/L provided a sensitivity=0.833, a specificity=0.568 and a ROC AUC=0.790 (95% confidence interval 0.692, 0.888).

Out-of-hospital-cardiac-arrest (OHCA) patients are patients that experience cardiac arrest while not admitted to a hospital, i.e. “out-of-hospital”. Approximately 50-89% of OHCA patients die after admittance to the hospital. Also in this group of critically ill patients the metabolic biomarker; succinic acid, could specifically identify the patients with increased risk of (early) death. A succinic acid cut-off level of 7 μmol/L provided a sensitivity=0.750, a specificity=0.709, and a ROC AUC=0.760 (95% confidence interval 0.632, 0.888).

The inventors have identified the mechanism responsible for the high death rate and have found that it is related to a toxic hyperactivation of the sympathetic nervous system in critically ill patients not surviving 72 hours from admission with approximately a doubling of the levels of circulating catecholamines. The cardiac effects of toxic levels of catecholamines have been described extensively in the literature and exposure of human myocardium to high catecholamine levels has long been recognized as inducing myocardial necrosis (Rona et al., 1985). The catecholamine-induced cardiomyopathy is a well-recognized and potentially life-threatening complication in pheochromocytoma and paragangliomas and play a critical role in the pathogenesis of the acute and severe heart failure entitled Takotsubo cardiomyopathy (TC) (Wittstein et al., 2005, Ansari et al., 2018, Otusanya et al., 2015, Giavarini et al., 2013). Furthermore, stress induced myocardial stunning or “broken heart” has been reported where circulating epinephrine levels were 4 times higher than in patients experiencing acute myocardial infarction (1.275 pg/ml vs. 376 pg/ml) suggesting this pathology also is present in the trauma, sepsis and OHCA TOX patients investigated who had approximately 2 times higher circulating catecholamines levels compared to non-TOX patients (Abraham et al., 2009). Similarly, accidental epinephrine intoxication causes acute coronary spasm with significant myocardial ischemia and infarction, cardiac arrhythmias, transient left ventricular dysfunction, pulmonary edema and high mortality (Abraham et al., 2009).

In critically ill patients, the Toxic Catecholamine Syndrome described above is particularly challenging as it is combined with life-threatening hemodynamic instability that poses excessive stress on the myocardium (Johansson et al., 2017). A consistent feature of Toxic Catecholamine Syndrome is the inability to maintain adequate blood pressure and this is particularly catastrophic in patients with traumatic brain injury where high blood pressure is a prerequisite to ensure adequate perfusion pressure to the brain and, thereby, oxygen delivery to the cerebral cells (Rakhit et al., 2021). It should be emphasized that identifying patients with Toxic Catecholamine Syndrome is pivotal as the current standard management of shock induced hypotension is administration of high doses of catecholamines, which in this subpopulation of patients, further propagates the disastrous cardiac effects contributing to the high mortality observed. Importantly, this toxic condition can be reversed by pharmacological beta-adrenergic blockage.

In one aspect of the present invention, the invention relates to a medicament which inhibits catecholamine release and/or blocks adrenergic receptors for use in the treatment of a subject suffering from Toxic Catecholamine Syndrome.

In one aspect of the present invention, the invention relates to a medicament which inhibits catecholamine release and/or blocks adrenergic receptors for use in the treatment of a subject having an elevated succinic acid level compared to a reference level of succinic acid.

In one aspect of the present invention, the invention relates to the use of a medicament which inhibits catecholamine release and/or blocks adrenergic receptors in the manufacture of a medicament for the treatment of a subject suffering from Toxic Catecholamine Syndrome.

In one aspect of the present invention, the invention relates to a method of treatment of a subject suffering from Toxic Catecholamine Syndrome, wherein said method comprises administering a medicament which inhibits catecholamine release and/or blocks adrenergic receptors to said subject.

A subject suffering from Toxic Catecholamine Syndrome can be identified by measuring the level of succinic acid in a sample obtained from said subject, wherein a subject suffering from Toxic Catecholamine Syndrome has an increased level of succinic acid compared to a reference level as described herein. Thus, in one embodiment, the present invention relates to a method of treatment of a subject having an elevated succinic acid level compared to a reference level, such as a succinic acid level above a cut-off as defined herein.

In some embodiments, the succinic acid level may be measured at the site of an accident, on route to the hospital or upon arrival at the emergency department or at arrival in the intensive care unit (ICU) to provide for early identification of Toxic Catecholamine Syndrome in a subject and initiation of treatment. Thus, in one embodiment, a measurement of the level of succinic acid in a sample may be obtained within 3 hours, such as within 2 hours, such as within 1 hour, such as within 30 minutes, such as within 15 minutes, such as within 10 minutes, such as within 5 minutes, such as within 3 minutes, such as within 2 minutes of obtaining a sample from a critically ill subject.

Thus, it would be possible to treat a subject suffering from Toxic Catecholamine Syndrome within 3 hours, such as within 2 hours, such as within 1 hour, such as within 30 minutes, such as within 25 minutes, such as within 20 minutes, such as within 15 minutes, such as within 10 minutes, such as within 5 minutes from disease onset.

For a trauma patient, disease onset would be the time of the traumatic injury occurring. For a sepsis patient, the disease onset would be the debut of systemic infection. For an OHCA patient, the disease onset would be the time of occurrence of the cardiac arrest event. A person skilled in the art will thus appreciate that it is possible to treat a critically ill subject as described herein already at the site of the accident for trauma patients/OHCA patients, on route to the hospital or immediately upon arrival at the hospital emergency department or ICU. For a sepsis patient, appropriate treatment of toxic catecholamine syndrome can likewise be initiated shortly after debut of systemic infection.

The term “Toxic Catecholamine Syndrome” is used throughout this document. The term is used in reference to critically ill subjects having toxic hyperactivation of the sympathetic nervous system. The inventors have found that these patients have elevated succinic acid levels and that they may benefit from treatment with a medicament which inhibits catecholamine release and/or blocks adrenergic receptors.

In one embodiment of the present invention, the medicament that inhibits catecholamine release and/or blocks adrenergic receptors is for administration to a subject having an elevated succinic acid level compared to a reference level of succinic acid.

In one embodiment, said elevated succinic acid level is at least 1.2 fold higher compared to a reference level, such as at least 1.3, such as at least 1.4, such as at least 1.5, such as at least 1.6, such as at least 1.7, such as at least 1.8, such as at least 1.9, such as at least 2.0, such as at least 2.5, such as at least 3.0, such as at least 3.5, such as at least 4.0, such as at least 4.5, such as at least 5.0, such as at least 5.5, such as least 6.0, such as at least 6.5, such as at least 7.0, such as at least 7.5, such as at least 8.0, such as at least 8.5, such as at least 9.0, such as at least 9.5, such as at least 10.0.

In one embodiment, the elevated succinic acid level is at least 1.4 fold higher compared to a reference level.

In one embodiment, the elevated succinic acid level is at least 1.5 fold higher compared to a reference level.

In one embodiment, the elevated succinic acid level is at least 1.6 fold higher compared to a reference level.

In one embodiment, the elevated succinic acid level is at least 1.7 fold higher compared to a reference level.

In one embodiment, the elevated succinic acid level is at least 1.8 fold higher compared to a reference level.

In one embodiment, the elevated succinic acid level is at least 1.9 fold higher compared to a reference level.

In one embodiment, the elevated succinic acid level is at least 2.0 fold higher compared to a reference level.

In one embodiment, the elevated succinic acid level is at least 2.5 fold higher compared to a reference level.

In one embodiment, the elevated succinic acid level is at least 3.0 fold higher compared to a reference level.

In one embodiment, the elevated succinic acid level is at least 3.5 fold higher compared to a reference level.

In one embodiment, the elevated succinic acid level is at least 4.0 fold higher compared to a reference level.

In one embodiment, the subjects are critically ill with shock. The term “shock” is a condition where a subject suffers from too low blood pressure to be able to maintain a normal oxygenation.

As used herein, “reference level” refers to the average level, the median level or concentration of a certain factor, such as succinic acid, in a population of critically ill patients, such as trauma, sepsis or OHCA patients that are to be admitted, or have been admitted, to an emergency department or ICU. A “reference subject” is a critically ill patient, such as a trauma, sepsis or OHCA patient that is to be admitted, or has been admitted, to an emergency department or ICU.

In one embodiment, the subject is a trauma patient, i.e. a subject who has suffered a traumatic injury and that is to be admitted, or has been admitted, to a Trauma Centre. In one embodiment, the subject is suffering from brain injury, such as traumatic brain injury.

In one embodiment, the reference level is obtained from a population of trauma patients, for example wherein the reference level is the average succinic acid level in a population of trauma patients, such as the average succinic acid level in a population of trauma patients, such as trauma patients that do not suffer from Toxic Catecholeamine Syndrome.

In one embodiment, the medicament which inhibits catecholamine release and/or blocks adrenergic receptors is for administration to a trauma patient having a succinic acid level above a cut-off value of between 12 μmol/L and 40 μmol/L, such as between 13 μmol/L and 40 μmol/L, such as between 14 μmol/L and 40 μmol/L, such as between 15 μmol/L and 40 μmol.

In one embodiment, the succinic acid cut-off value for trauma patients is between 12 μmol/L and 30 μmol/L, such as between 13 μmol/L and 30 μmol/L, such as between 14 μmol/L and 30 μmol/L, such as between 15 μmol/L and 30 μmol/L.

In one embodiment, the succinic acid cut-off value for trauma patients is between 12 μmol/L and 20 μmol/L, such as between 13 μmol/L and 20 μmol/L, such as between 14 μmol/L and 20 μmol/L, such as between 15 μmol/L and 20 μmol/L.

In one embodiment, the medicament which inhibits catecholamine release and/or blocks adrenergic receptors is for administration to a trauma patient having a succinic acid level above about 15 μmol/L.

In one embodiment, the subject is a sepsis patient.

In one embodiment, the reference level is obtained from a population of sepsis patients, for example wherein the reference level is the average succinic acid level in a population of sepsis patients, such as the average succinic acid level in a population of sepsis patients, such as sepsis patients that do not suffer from Toxic Catecholamine Syndrome.

In one embodiment, the medicament which inhibits catecholamine release and/or blocks adrenergic receptors is administered to a sepsis patient having a succinic acid level above a cut-off value of between 8 μmol/L and 40 μmol/L, such as between 9 μmol/L and 40 μmol/L, such as between 10 μmol/L and 40 μmol/L.

In one embodiment, the medicament which inhibits catecholamine release and/or blocks adrenergic receptors is administered to a sepsis patient having a succinic acid level above a cut-off value of between 10 μmol/L and 30 μmol/L, such as between 10 μmol/L and 20 μmol/L, such as between 10 μmol/L and 15 μmol/L.

In one embodiment, the medicament which inhibits catecholamine release and/or blocks adrenergic receptors is administered to a sepsis patient having a succinic acid level above about 10 μmol/L.

In one embodiment the subject is an OHCA patient.

In one embodiment, the reference level is obtained from a population of OHCA patients, for example wherein the reference level is the average succinic acid level in a population of OHCA patients, such as the average succinic acid level in a population of OHCA patients, such as OHCA patients that do not suffer from Toxic Catecholamine Syndrome.

In one embodiment, the medicament which inhibits catecholamine release and/or blocks adrenergic receptors is administered to an OHCA patient having a succinic acid level above a cut-off value of between 5 μmol/L and 40 μmol/L, such as between 6 μmol/L and 40 μmol/L, such as between 7 μmol/L and 40 μmol/L.

In one embodiment the medicament which inhibits catecholamine release and/or blocks adrenergic receptors is administered to an OHCA patient having a succinic acid level above about 7 μmol/L.

In one embodiment, the subject is an adult. In other embodiments the subject is an infant, a child or an adolescent.

In one embodiment, the subject having an elevated succinic acid level further has an elevated level of one or more catecholamines. In one embodiment, the catecholamines are adrenaline and/or noradrenaline. A person skilled in the art will recognize there is a large difference in adrenaline levels between patients with and without Toxic Catecholamine Syndrome. However, the inventors have found that it is not possible to correctly identify all patients with an increased risk of early death solely based on the levels of catecholamines. Therefore, an elevated succinic acid level is considered a superior biomarker for identification and treatment of toxic catecholamine syndrome subjects.

In the context of the invention, the term “catecholamine” refers to hormones released by the adrenal glands, which are part of the sympathetic nervous system and contain a catechol or 3,4-dihydroxyphenyl group. They have more specifically the distinct structure of a benzene ring with two hydroxyl groups, an intermediate ethyl chain and a terminal amine group. Catecholamines include in particular adrenaline (also called epinephrine), noradrenaline (also called norepinephrine), and dopamine, all of which are produced from phenylalanine and tyrosine.

Two catecholamines; noradrenaline and dopamine, act as neuromodulators in the central nervous system (CNS) and as hormones in the blood circulation. Adrenaline acts as a neurotransmitter involved in regulating visceral functions (e.g., respiration), and plays an important role in the fight-or-flight response by increasing blood flow to muscles, output of the heart, pupil dilation response and blood sugar level.

In one embodiment of the present invention, the medicament which inhibits catecholamine release and/or blocks adrenergic receptors is selected from an adrenergic receptor antagonist, a catecholamine synthesis antagonist and a catecholamine release inhibitor.

In one embodiment, the medicament blocks adrenergic receptors.

The adrenergic receptors, also called adrenoceptors, are a class of G protein-coupled receptors with catecholamines, such as adrenaline and noradrenaline produced by the body, as substrates. There are also several medications that targets adrenergic receptors, such as beta-agonists or beta-blockers.

As used herein “antagonist”, “blocker”, “inhibitor” are used interchangeably throughout the document to refer to an agent that decreases or suppresses a biological activity, such as to repress an activity of a receptor, such as a beta-adrenergic or alpha-adrenergic receptors. Similarly, the terms “blocks” and “inhibits” are used interchangeably throughout the document.

In one embodiment of the present invention, the medicament is a beta-blocker.

In one embodiment, the beta-blocker is a non-selective beta-blocker, such as bucindolol, carteolol, carvedilol, labetalol, nadolol, oxprenolol, penbutolol, pindolol, propranolol, sotalol or timolol.

In one embodiment, the beta-blocker is a selective beta-blocker. In one embodiment of the present invention, the selective beta-blocker is a beta-1 selective beta-blocker, such as acebutolol, atenolol, betaxolol, bisoprolol, celiprolol, esmolol, metoprolol or nebivolol. In yet another embodiment, the selective beta-blocker is a beta-2 selective beta-blocker, such as butaxamine.

In a particularly preferred embodiment, the selective beta-blocker is esmolol. In another preferred embodiment, the selective beta-blocker is landiolol.

Beta-blockers are a class of medications that are predominantly used to manage abnormal heart rhythms, and to protect the heart from a second myocardial infarction after a first heart attack. They are also widely used to treat hypertension, although they are no longer the first choice for initial treatment of most patients. Beta-blockers are competitive antagonists that block the receptor sites for the endogenous catecholamines adrenaline and noradrenaline on adrenergic beta-receptors, of the sympathetic nervous system, which mediates the fight-or-flight response. Some block activation of all types of beta-adrenergic receptors and others are selective for one of the three known types of beta-adrenergic receptors.

The term “beta-blocker” or “beta-adrenergic receptor antagonist” as used herein, are synonymous, and refers to beta-receptor blocking agent, beta adrenergic receptor blocking agent, beta blocking agent, beta-blocking agent, beta-blocking agent or beta-adrenergic receptor blocking agent or any other denomination indicating a chemical that inhibits the binding of agonists, natural or artificial, to beta-adrenergic receptors of any type (beta-1, beta-2, beta-3 or others). Suitable beta-blockers include compounds selected from acebutolol, atenolol, betaxolol, bisoprolol, bucindolol, butaxamine, carteolol, carvedilol, celiprolol, esmolol, labetalol, metoprolol, nadolol, nebivolol, oxprenolol, penbutolol, pindolol, propranolol, sotalol and timolol. Where the beta-blocker is an acid or base or otherwise capable of forming pharmaceutically acceptable salts or prodrugs, these forms are considered to be encompassed herein, and it is understood that the compounds may be administered in free form or in the form of a pharmaceutically acceptable salt or a prodrug, such as a physiologically hydrolyzable and acceptable ester. For example, metoprolol is suitably administered as its tartrate salt, propranolol is suitably administered as the hydrochloride salt, and so forth.

In one embodiment of the present invention, the medicament is not an alpha-blocker.

The term “alpha-blocker” or “alpha-adrenergic receptor antagonist” as used herein, are synonymous, and refers to an alpha-receptor blocking agent, alpha adrenergic receptor blocking agent, alpha blocking agent, alpha-blocking agent, alpha-blocking agent or alpha-adrenergic receptor blocking agent or any other denomination indicating a chemical that inhibits the binding of agonists, natural or artificial, to alpha-adrenergic receptors of any type (alpha-1 or alpha-2, or others). Alpha-blockers can treat a small range of diseases such as hypertension, Raynaud's disease, benign prostatic hyperplasia and erectile dysfunction. In general terms, these treatments function by binding to alpha-adrenergic receptors in the arteries and smooth muscle. Ultimately, depending on the type of alpha-adrenergic receptor, this relaxes the smooth muscle or blood vessels, which increases fluid flow in these entities. Examples of alpha-blockers include compounds selected from alfuzosin, doxazosin, prazosin, tamsulosin, terazosin, silodosin, atipamezole, idazoxan, mirtazapine or yohimbine.

In one embodiment of the present invention, the medicament is a catecholamine synthesis antagonist. In another embodiment, the catecholamine synthesis antagonist is a tyrosine hydroxylase inhibitor. In yet another embodiment, the tyrosine hydroxylase inhibitor is metyrosine.

The term “catecholamine synthesis antagonist” as used herein, refers to a compound capable of inhibiting the endogenous synthesis of catecholamines. The term “metyrosine” as used herein, is synonymous with metirosine, α-methyltyrosine, alpha-methyltyrosine, alpha-methyl-p-tyrosine or any other methylated tyrosine capable of inhibiting tyrosine hydroxylase. Tyrosine hydroxylases catalyses the rate limiting step in the synthesis of catecholamines, hence their inhibition results in inhibition of catecholamine synthesis.

In one embodiment of the present invention, the medicament is a catecholamine release inhibitor.

In one embodiment, the catecholamine release inhibitor is a natriuretic peptide.

In one embodiment, the natriuretic peptide is an atrial natriuretic peptide.

In one embodiment, the natriuretic peptide is an atrial natriuretic peptide homologue, such as ularitide.

In one embodiment, natriuretic peptide is a ventricular natriuretic peptide.

In one embodiment, the natriuretic peptide is a recombinant ventricular natriuretic peptide, such as nesiritide.

The term “natriuretic peptide” refers to a peptide that has the biological activity of promoting natriuresis, diuresis, and vasodilation. Assays for testing such activity are known in the art, e.g., as described in U.S. Pat. Nos. 4,751,284 and 5,449,751. Examples of natriuretic peptides include, but are not limited to, atrial natriuretic peptide (ANP(99-126)), brain natriuretic peptide (BNP), C-type natriuretic peptide (CNP), Dendroaspis natriuretic peptide (DNP), urodilatin (URO, or ularitide), and any fragments of the prohormone ANP(1-126) or BNP precursor polypeptide that retains the vasodilating, natriuretic, or diuretic activity. For further description of exemplary natriuretic peptides and their use or preparation, see, e.g., U.S. Pat. Nos. 4,751,284, 4,782,044, 4,895,932, 5,449,751, 5,461,142, 5,571,789, and 5,767,239. See also, Ha et al., Regul. Pept. 133(1-3): 13-19, 2006. The term “atrial natriuretic peptide” or “ANP(99-126)” refers to a 28-amino acid peptide hormone, which is derived from the same polypeptide precursor, ANP(1-126). The term “ventricular natriuretic peptide” is synonymous with brain natriuretic peptide (BNP) and B-type natriuretic peptide.

In one embodiment, said critically ill subject is treated with a combination treatment of the standard treatment for said acute critical illness, and the medicament as described herein.

In one embodiment of the present invention, the level of succinic acid is measured in a biological sample obtained from the subject, such as a blood sample. In one embodiment, the sample is whole blood, i.e. unfractionated blood. The use of unfractionated blood is particularly useful for measuring succinic acid levels via a point-of-care test at the site of accident or on route to the hospital since no laboratory facilities are required and the lack of sample preparation will allow for early identification and initiation of treatment of a subject predicted to suffer from Toxic Catecholamine Syndrome. In one embodiment, the sample is a plasma sample.

Succinic acid may be measured by any method known to the person of skill. Exemplary methods for measuring succinic acid include colorimetric assays or mass spectrometry, such as gas chromatography mass spectrometry (GC-MS), a colorimetric assay, by aptamer or an ELISA. Succinic acid may e.g. be measured by a point-of-care test allowing for a rapid, precise result without the need for time-consuming sample preparation and/or laboratory facilities.

In one aspect of the invention, the invention relates to a method of identifying a subject who is likely to benefit from treatment with a medicament which inhibits catecholamine release and/or blocks adrenergic receptors, wherein said method comprises the steps:
(a) providing a sample obtained from said subject;
(b) determining the concentration of succinic acid in said sample;
   wherein a subject having an elevated succinic acid level compared to a reference level is likely to benefit from treatment with a medicament capable of inhibiting catecholamine release and/or blocking adrenergic receptors.

In one aspect of the present invention, said medicament is used in the manufacture of a medicament for the treatment of a patient identified as likely to benefit from treatment with a medicament which inhibits catecholamine release and/or blocks adrenergic receptors according to the methods herein.

In one embodiment, the present invention relates to a method of treatment of a critically ill subject, said method comprising:
(a) providing a sample obtained from said subject;
(b) determining the concentration of succinic acid in said sample;
(c) comparing the concentration of succinic acid in said sample with a reference level, wherein a subject having an elevated succinic acid level compared to a reference level is likely to benefit from treatment with a medicament which inhibits catecholamine release and/or blocks adrenergic receptors; and
(d) administering an effective amount of a medicament which inhibits catecholamine release and/or blocks adrenergic receptors to the subject.

Samples, reference levels, cutoffs, patient groups and medicaments applied in relation to the method of treatment may be as described elsewhere herein.

### Items

1. A medicament which inhibits catecholamine release and/or blocks adrenergic receptors for use in the treatment of a subject suffering from Toxic Catecholamine Syndrome.
2. The medicament for use according to item 1, wherein the medicament which inhibits catecholamine release and/or blocks adrenergic receptors is administered to a subject having an elevated succinic acid level compared to a reference level of succinic acid.
3. The medicament for use according to item 2, wherein the succinic acid level is at least 1.2 fold higher compared to a reference level, such as at least 1.3, such as at least 1.4, such as at least 1.5, such as at least 1.6, such as at least 1.7, such as at least 1.8, such as at least 1.9, such as at least 2.0, such as at least 2.5, such as at least 2.6, such as at least 2.7, such as at least 2.8, such as at least 2.9, such as at least 3.0, such as at least 3.1, such as at least 3.2, such as at least 3.3, such as at least 3.4, such as at least 3.5, such as at least 3.6, such as at least 3.7, such as at least 3.8, such as at least 3.9, such as at least 4.0 fold higher compared to a reference level.
4. The medicament for use according to any one of the preceding items, wherein the subject is a critically ill subject with shock.
5. The medicament for use according to any one of the preceding items, wherein the subject is a trauma patient, a sepsis patient or an out-of-hospital-cardiac arrest (OHCA) patient.
6. The medicament for use according to any one of the preceding items, wherein the subject is a trauma patient.
7. The medicament for use according to item 6, wherein the reference level is obtained from a population of trauma patients, for example wherein the reference level is the average succinic acid level in a population of trauma patients, such as the average succinic acid level in a population of trauma patients that do not suffer from Toxic Catecholamine Syndrome.
8. The medicament for use according to any one of the preceding items, wherein the medicament which inhibits catecholamine release and/or blocks adrenergic receptors is administered to a subject having a succinic acid level above a cut-off value of between 12 μmol/L and 40 μmol/L, such as between 13 μmol/L and 40 μmol/L, such as between 14 μmol/L and 40 μmol/L, such as between 15 μmol/L and 40 μmol/L.
9. The medicament for use according to any one of the preceding items, wherein the medicament which inhibits catecholamine release and/or blocks adrenergic receptors is administered to a subject having a succinic acid level above about 15 μmol/L.
10. The medicament for use according to any one of items 1 to 5, wherein the subject is a sepsis patient.
11. The medicament for use according to any one of items 1 to 5 or 10, wherein the reference level is obtained from a population of sepsis patients, for example wherein the reference level is the average succinic acid level in a population of sepsis patients, such as the average succinic acid level in a population of sepsis patients that do not suffer from Toxic Catecholamine Syndrome.
12. The medicament for use according to any one of items 1 to 5 or 10 to 11, wherein the medicament which inhibits catecholamine release and/or blocks adrenergic receptors is administered to a subject having a succinic acid level above a cut-off value of between 8 μmol/L and 40 μmol/L, such as between 9 μmol/L and 40 μmol/L, such as between 10 μmol/L and 40 μmol/L.
13. The medicament for use according to any one of items 1 to 5 or 10 to 12, wherein the medicament which inhibits catecholamine release and/or blocks adrenergic receptors is administered to a subject having a succinic acid level above about 10 μmol/L.
14. The medicament for use according to any one of items 1 to 5, wherein the subject is an OHCA patient.
15. The medicament for use according to any one of items 1 to 5 or 14, wherein the reference level is obtained from a population of OHCA patients, for example wherein the reference level is the average succinic acid level in a population of OHCA patients, such as the average succinic acid level in a population of OHCA patients that do not suffer from Toxic Catecholamine Syndrome.
16. The medicament for use according to any one of items 1 to 5 or 14 to 15, wherein the medicament which inhibits catecholamine release and/or blocks adrenergic receptors is administered to a subject having a succinic acid level above a cut-off value of between 5 μmol/L and 40 μmol/L, such as between 6 μmol/L and 40 μmol/L, such as between 7 μmol/L and 40 μmol/L.
17. The medicament for use according to any one of items 1 to 5 or 14 to 16, wherein the medicament which inhibits catecholamine release and/or blocks adrenergic receptors is administered to a subject having a succinic acid level above about 7 μmol/L.
18. The medicament for use according to any one of the preceding items, wherein the subject having an elevated succinic acid level further has an elevated level of one or more catecholamines.
19. The medicament for use according to item 18, wherein the catecholamines are adrenaline and/or noradrenaline.
20. The medicament for use according to any one of the preceding items, wherein the medicament which inhibits catecholamine release and/or blocks adrenergic receptors is selected from an adrenergic receptor antagonist, a catecholamine synthesis antagonist and a catecholamine release inhibitor.
21. The medicament for use according to any one of the preceding items, wherein the medicament blocks adrenergic receptors.
22. The medicament for use according to any of the preceding items, wherein the medicament is a beta-blocker.
23. The medicament for use according to item 22, wherein the beta-blocker is a non-selective beta-blocker, such as bucindolol, carteolol, carvedilol, labetalol, nadolol, oxprenolol, penbutolol, pindolol, propranolol, sotalol or timolol.
24. The medicament for use according to item 22, wherein the beta-blocker is a selective beta-blocker.
25. The medicament for use according to item 24, wherein the selective beta-blocker is a beta-1 selective beta-blocker, such as acebutolol, atenolol, betaxolol, bisoprolol, celiprolol, esmolol, landiolol, metoprolol or nebivolol, preferably wherein the beta-1 selective beta-blocker is esmolol or landiolol.
26. The medicament for use according to item 24, wherein the selective beta-blocker is a beta-2 selective beta-blocker, such as butaxamine.
27. The medicament for use according to any one of the preceding items, wherein the medicament is not an alpha-blocker.
28. The medicament for use according to any one of items 1 to 20, wherein the medicament is a catecholamine synthesis antagonist.
29. The medicament for use according to item 28, wherein the catecholamine synthesis antagonist is a tyrosine hydroxylase inhibitor.
30. The medicament for use according to item 29, wherein the tyrosine hydroxylase inhibitor is metyrosine.
31. The medicament for use according to any one of items 1 to 20, wherein the medicament is a catecholamine release inhibitor.
32. The medicament for use according to item 31, wherein the catecholamine release inhibitor is a natriuretic peptide.
33. The medicament for use according to item 32, wherein the natriuretic peptide is an atrial natriuretic peptide.
34. The medicament for use according to item 32, wherein the natriuretic peptide is an atrial natriuretic peptide homologue, such as ularitide.
35. The medicament for use according to item 32, wherein the natriuretic peptide is a ventricular natriuretic peptide.
36. The medicament for use according to item 32, wherein the natriuretic peptide is a recombinant ventricular natriuretic peptide, such as nesiritide.
37. The medicament for use according to any one of items 2 to 36, wherein the level of succinic acid is measured in a biological sample obtained from the subject, such as a blood sample.
38. The medicament for use according to item 31, wherein the blood sample is whole blood or plasma.
39. The medicament for use according to any one of the preceding items, wherein succinic acid is measured by mass spectrometry, such as by gas chromatography mass spectrometry (GC-MS), by aptamer, or by a colorimetric assay.
40. The medicament for use according to any one of the preceding items, wherein succinic acid is measured by a point-of-care test.
41. The medicament for use according to any one of the preceding items, wherein the medicament is administered to a subject within 3 hours, such as within 2 hours, such as within 1 hour, such as within 30 minutes, such as within 25 minutes, such as within 20 minutes, such as within 15 minutes, such as within 10 minutes, such as within 5 minutes from disease onset and/or the occurrence of the traumatic injury.
42. The medicament for use according to any one of the preceding items, wherein said medicament is administered in combination with standard therapy for a critically ill subject.
43. The medicament for use according to item 42, wherein the critically ill subject is a trauma patient.
44. The medicament for use according to item 42, wherein the critically ill subject is a sepsis patient.
45. The medicament for use according to item 42, wherein the critically ill subject is an OHCA patient.
46. A method of identifying a subject who is likely to benefit from treatment with a medicament which inhibits catecholamine release and/or blocks adrenergic receptors, wherein said method comprises the steps:
   (a) providing a sample obtained from said subject;
   (b) determining the concentration of succinic acid in said sample;
   wherein a subject having an elevated succinic acid level compared to a reference level is likely to benefit from treatment with a medicament capable of inhibiting catecholamine release and/or blocking adrenergic receptors.
47. The method according to item 46, wherein the sample is a blood sample, such as whole blood or plasma.
48. The method according to any one of items 46-47, wherein the subject is a critically ill subject with shock, such as a trauma patient, a sepsis patient or an OHCA patient.
49. The method according to any one of items 46-48, wherein the elevated succinic acid level is at least 1.2 fold higher compared to a reference level, such as at least 1.3, such as at least 1.4, such as at least 1.5, such as at least 1.6, such as at least 1.7, such as at least 1.8, such as at least 1.9, such as at least 2.0, such as at least 2.5, such as at least 2.6, such as at least 2.7, such as at least 2.8, such as at least 2.9, such as at least 3.0, such as at least 3.1, such as at least 3.2, such as at least 3.3, such as at least 3.4, such as at least 3.5, such as at least 3.6, such as at least 3.7, such as at least 3.8, such as at least 3.9, such as at least 4.0 fold higher compared to a reference level.
50. The method according to any one of items 46-49, wherein the subject is a trauma patient.
51. The method according to item 50, wherein the reference level is obtained from a population of trauma patients, for example wherein the reference level is the average succinic acid level in a population of trauma patients, such as the average succinic acid level in a population of trauma patients that do not suffer from Toxic Catecholamine Syndrome.
52. The method according to any one of items 50 to 51, wherein the medicament which inhibits catecholamine release and/or blocks adrenergic receptors is administered to a subject having a succinic acid level above a cut-off value of between 12 μmol/L and 40 μmol/L, such as between 13 μmol/L and 40 μmol/L, such as between 14 μmol/L and 40 μmol/L, such as between 15 μmol/L and 40 μmol/L.
53. The method according to any one of items 50 to 52, wherein the medicament which inhibits catecholamine release and/or blocks adrenergic receptors is administered to a subject having a succinic acid level above about 15 μmol/L.
54. The method according to any one of items 46 to 49, wherein the subject is a sepsis patient.
55. The method according to item 54, wherein the reference level is obtained from a population of sepsis patients, for example wherein the reference level is the average succinic acid level in a population of sepsis patients, such as the average succinic acid level in a population of sepsis patients that do not suffer from Toxic Catecholamine Syndrome.
56. The method according to any one of items 54 to 55, wherein the medicament which inhibits catecholamine release and/or blocks adrenergic receptors is administered to a subject having a succinic acid level above a cut-off value of between 8 μmol/L and 40 μmol/L, such as between 9 μmol/L and 40 μmol/L, such as between 10 μmol/L and 40 μmol/L.
57. The method according to any one of items 54 to 56, wherein the medicament which inhibits catecholamine release and/or blocks adrenergic receptors is administered to a subject having a succinic acid level above about 10 μmol/L.
58. The method according to any one of items 46 to 49, wherein the subject is an OHCA patient.
59. The method according to item 58, wherein the reference level is obtained from a population of OHCA patients, for example wherein the reference level is the average succinic acid level in a population of OHCA patients, such as the average succinic acid level in a population of OHCA patients that do not suffer from Toxic Catecholamine Syndrome.
60. The method according to any one of items 58 to 59, wherein the medicament which inhibits catecholamine release and/or blocks adrenergic receptors is administered to a subject having a succinic acid level above a cut-off value of between 5 μmol/L and 40 μmol/L, such as between 6 μmol/L and 40 μmol/L, such as between 7 μmol/L and 40 μmol/L.
61. The method according to any one of items 58 to 60, wherein the medicament which inhibits catecholamine release and/or blocks adrenergic receptors is administered to a subject having a succinic acid level above about 7 μmol/L.
62. The method according to any one of items 46-61, wherein the subject likely to benefit from treatment further has elevated levels of catecholamines.
63. The method according to item 62, wherein the catecholamines are adrenaline and/or noradrenaline.
64. The method according to any one of items 46-63, wherein the subject having elevated succinic acid levels has an increased risk of early death.
65. The method according to any one of items 46-64, wherein the medicament which inhibits catecholamine release and/or blocks adrenergic receptors is selected from adrenergic receptor antagonists, catecholamine synthesis antagonist and catecholamine release inhibitors.
66. The method according to any one of items 46-65, wherein the medicament blocks adrenergic receptors.
67. The method according to any one of items 46-66, wherein the medicament is a beta-blocker.
68. The method according to item 67, wherein the beta-blocker is a non-selective beta-blocker, such as bucindolol, carteolol, carvedilol, labetalol, nadolol, oxprenolol, penbutolol, pindolol, propranolol, sotalol or timolol.
69. The method according to item 68, wherein the beta-blocker is a selective beta-blocker.
70. The method according to item 69, wherein the selective beta-blocker is a beta-1 selective beta-blocker, such as acebutolol, atenolol, betaxolol, bisoprolol, celiprolol, esmolol, landiolol, metoprolol or nebivolol.
71. The method according to item 69, wherein the selective beta-blocker is a beta-2 selective beta-blocker, such as butaxamine.
72. The method according to any one of items 46-71, wherein the medicament is not an alpha-blocker.
73. The method according to any one of items 46-65, wherein the medicament is a catecholamine synthesis antagonist.
74. The method according to item 73, wherein the catecholamine synthesis antagonist is a tyrosine hydroxylase inhibitor.
75. The method according to item 74, wherein the tyrosine hydroxylase inhibitor is metyrosine.
76. The method according to any one of items 46-65, wherein the medicament is a catecholamine release inhibitor.
77. The method according to item 76, wherein the catecholamine release inhibitor is a natriuretic peptide.
78. The method according to item 77, wherein the natriuretic peptide is an atrial natriuretic peptide.
79. The method according to item 77, wherein the natriuretic peptide is an atrial natriuretic peptide homologue, such as ularitide.
80. The method according to item 77, wherein the natriuretic peptide is a ventricular natriuretic peptide.
81. The method according to item 77, wherein the natriuretic peptide is a recombinant ventricular natriuretic peptide, such as nesiritide.
82. The method according to any one of items 46-81, comprising a further step of administering an amount of a medicament which inhibits catecholamine release and/or blocks adrenergic receptors to said subject.
83. Use of a medicament which inhibits catecholamine release and/or blocks adrenergic receptors in the manufacture of a medicament for the treatment of a subject suffering from Toxic Catecholamine Syndrome.

### EXAMPLES

### Example 1. Identification of a Biomarker for Early Deaths (i.e. within 72-Hours) in Trauma Patients

### Method

This study included 86 adult trauma patients (≥18 years) admitted directly from the scene of injury to a level 1 trauma center. Blood samples were collected immediately upon hospital admission in 3.2% citrated tubes. Tubes were kept at 5° C. prior to centrifugation at speed 3068, 1,800 g for 10 minutes at 5° C. at two times within one hour after obtaining the blood sample to separate plasma. Plasma was aliquoted and frozen at −80° C. for later analysis.

Gas chromatography mass spectrometry (GC-MS) was performed. The samples were pre-processed: First samples are filtrated to remove lipids and proteins using Phree filters: An aliquot of the sample is added to the Phree filter and mixed with acetonitrile containing 1% formic acid. The filters are centrifuged 1000×g/4° C./10 mins. Filtrate from the Phree filters were dried under nitrogen flow and reconstituted in ultra-pure water prior to derivatization using MCF (methyl chloroformate). Samples were derivatized with MCF using a slightly modified version of the protocol described by Smart et al., 2010. In short: reconstituted filtrate is derivatized as described by Smart et al. and the derivatization reaction are stopped by addition of chloroform. Subsequently the chloroform is injected on the GC. All samples were analyzed in a randomized order. Analysis was performed using gas chromatography (7890B, Agilent) coupled with a quadrupole mass spectrometry detector (5977B, Agilent). The system was controlled by ChemStation (Agilent). Raw data was converted to netCDF format using Chemstation (Agilent), before the data was imported and processed in Matlab R2018b (Mathworks, Inc.) using the PARADISe software described by Johnsen et al., 2017.

Succinic acid covering percentage was 100% i.e. no missing values. Succinic acid was quantified in concentration (μmol/L). Statistical analysis was performed using SPSS 25 (IBM Corporation, New York, NY). Receiver operating characteristic (ROC) curve analysis with the Youden index was used to determine the cut-off value of Succinic acid that maximized the sum of sensitivity and specificity as a predictor for 72-hours in-hospital mortality.

### Results

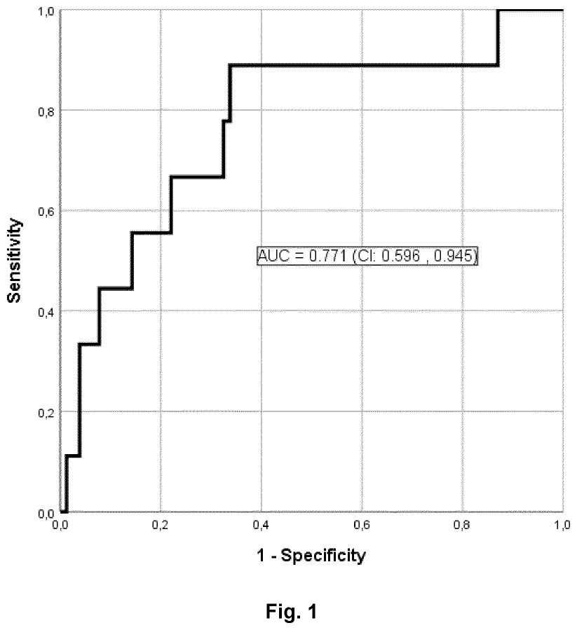

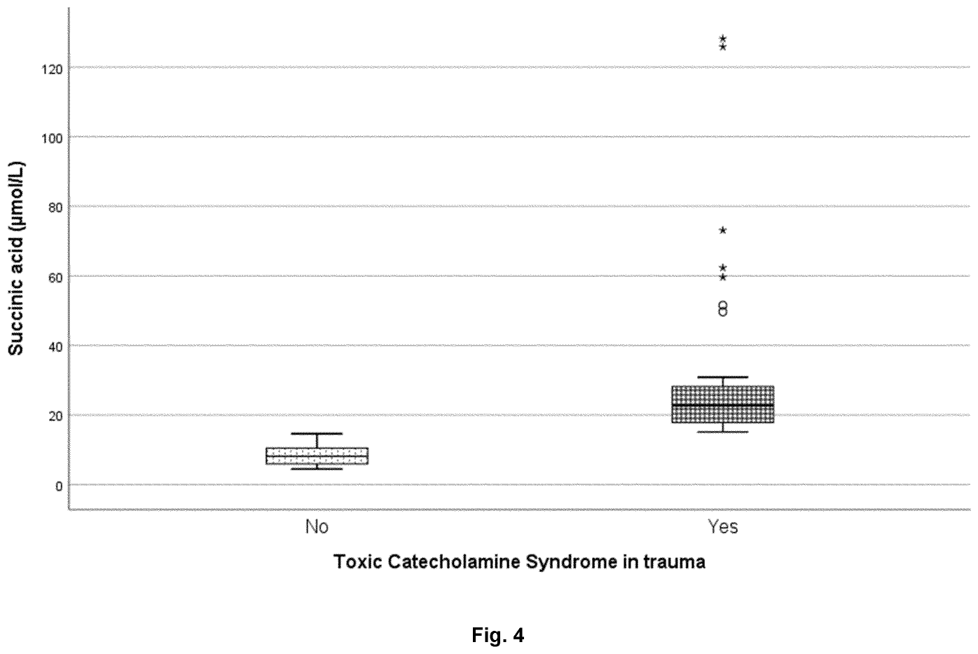

Succinic acid with a cut-off level above 15 μmol/L was able to predict the patients who die early, i.e. within 72 hours, with a sensitivity=0.889, a specificity=0.662, and a ROC AUC=0.771 (95% confidence interval 0.596, 0.945) (FIG. 1). A subsequent analysis showed that trauma patients with Toxic Catecholamine Syndrome had approximately 3.9 times higher levels of Succinic acid in their plasma compared to trauma patients without Toxic Catecholamine Syndrome (FIG. 4 and Table 1).

TABLE 1
Average levels of succinic acid (μM) in plasma of trauma patients

| | Overall | Non-TOX-trauma | TOX-trauma |
|---|---|---|---|
| | N = 86 | N = 51 | N = 35 |
| Succinic acid | 18.27 | 8.42 | 32.61 |
| (μM) | (7.20, 19.22) | (5.93, 10.50) | (17.79, 28.17) |

Mean (Interquartile range (IQR)) are reported.
TOX = Toxic Catecholamine Syndrome.

### Conclusions

Trauma patients suffering from Toxic Catecholamine Syndrome—identified as having a Succinic acid value above 15 μmol/L—have a significantly higher risk of early death (i.e. 72 hours). Since 40%-50% of total mortality for trauma patients occur within 72 hours, this finding is of major importance. In addition, trauma patients with Toxic Catecholamine Syndrome clearly had higher levels of Succinic acid, further supporting the use of Succinic acid as a biomarker for early death. This biomarker therefore enables identification of high-risk patients already at the scene of the accident or at the arrival to the trauma centre and thus early and life-saving treatment of these patients.

### Example 2. Identification of a Biomarker for Early Deaths (i.e. 72-Hours) in Sepsis Patients

### Method

This study included 179 adult sepsis patients (≥18 years) admitted to the ICU. Blood samples were collected immediately upon ICU admission in 3.2% citrated tubes. Tubes were kept at 5° C. prior to centrifugation at speed 3068, 1,800 g for 10 minutes at 5° C. at two times within one hour after obtaining the blood sample to separate plasma. Plasma was aliquoted and frozen at −80° C. for later analysis.

Gas chromatography mass spectrometry (GC-MS) was performed. The samples were pre-processed: First samples are filtrated to remove lipids and proteins using Phree filters: An aliquot of the sample is added to the Phree filter and mixed with acetonitrile containing 1% formic acid. The filters are centrifuged 1000×g/4° C./10 mins. Filtrate from the Phree filters were dried under nitrogen flow and reconstituted in ultra-pure water prior to derivatization using MCF (methyl chloroformate). Samples were derivatized with MCF using a slightly modified version of the protocol described by Smart et al., 2010. In short: reconstituted filtrate is derivatized as described by Smart et al. and the derivatization reaction are stopped by addition of chloroform. Subsequently the chloroform is injected on the GC. All samples were analyzed in a randomized order. Analysis was performed using gas chromatography (7890B, Agilent) coupled with a quadrupole mass spectrometry detector (5977B, Agilent). The system was controlled by ChemStation (Agilent). Raw data was converted to netCDF format using Chemstation (Agilent), before the data was imported and processed in Matlab R2018b (Mathworks, Inc.) using the PARADISe software described by Johnsen et al., 2017.

Succinic acid covering percentage was 100% i.e. no missing values. Succinic acid was quantified in concentration (μmol/L).

Statistical analysis was performed using SPSS 25 (IBM Corporation, New York, NY). Receiver operating characteristic (ROC) curve analysis with the Youden index was used to determine the cut-off value of Succinic acid that maximized the sum of sensitivity and specificity as a predictor for 72-hours in-hospital mortality.

### Results

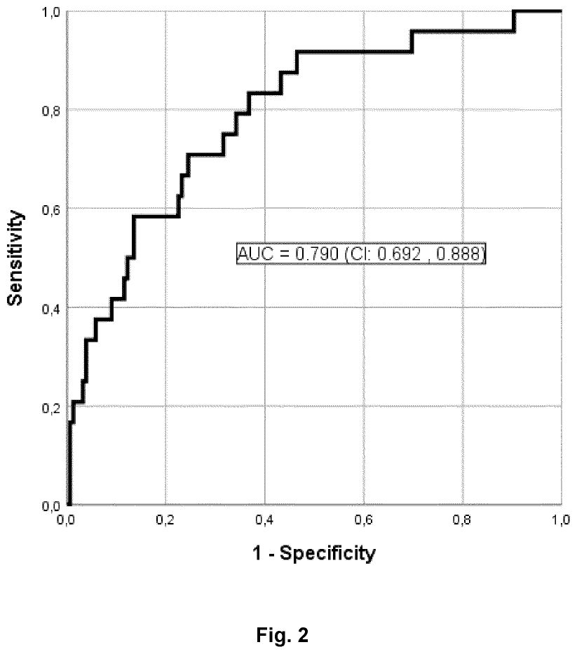

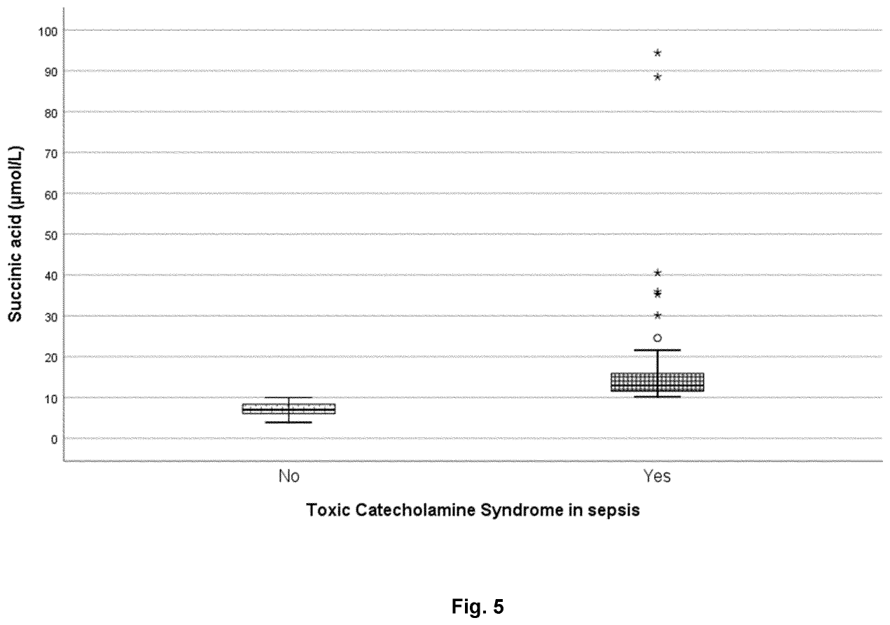

Succinic acid with a cut-off level above 10 μmol/L was able to predict the patients who die early, i.e. within 72 hours, with a sensitivity=0.833, a specificity=0.568, and a ROC AUC=0.790 (95% confidence interval 0.592, 0.888) (FIG. 2). An analysis showed that sepsis patients with Toxic Catecholamine Syndrome had approximately 2.3 times higher levels of Succinic acid in their plasma compared to trauma patients without Toxic Catecholamine Syndrome (FIG. 5 and Table 2).

TABLE 2
Average levels of succinic acid (μM) in plasma of sepsis patients

| | Overall | Non-TOX-sepsis | TOX-sepsis |
|---|---|---|---|
| | N = 179 | N = 92 | N = 87 |
| Succinic acid | 11.58 | 7.16 | 16.25 |
| (μM) | (6.93, 12.84) | (6.05, 8.32) | (11.53, 15.85) |

Mean (Interquartile range (IQR)) are reported.
TOX = Toxic Catecholamine Syndrome.

### Conclusions

Sepsis patients suffering from Toxic Catecholamine Syndrome—identified as having a Succinic acid value above 10 μmol/L—have a significantly higher risk of early death (i.e. 72 hours). Since 40% of total mortality for sepsis patients occur within 72 hours after debut of systemic infection, this finding is of major importance. In addition, sepsis patients with Toxic Catecholamine Syndrome clearly had higher levels of Succinic acid, further supporting the use of Succinic acid as a biomarker for early death. This biomarker therefore enables identification of high-risk patients already at admission to the ICU and thus early and life-saving treatment of these patients.

### Example 3. Trauma and Sepsis Patients with Toxic Catecholamine Syndrome Exhibit Higher Levels of Adrenaline and Noradrenaline Compared to the Rest of the Cohort

### Method

This experiment was performed on the same cohort as Example 1 and 2, and the same blood samples were used.

An Enzyme-linked immunosorbent assay (ELISA) was performed accordingly: The soluble biomarker of sympathoadrenal activation (adrenaline and noradrenaline) were measured according to the manufacturer's recommendation (2-CAT ELISA, Labor Diagnostica Nord GmbH). The covering percentage of adrenaline and noradrenaline were 100% for trauma patients i.e. no missing values and 99% for sepsis patients i.e. 2 missing values. The aim was to compare adrenaline and noradrenaline levels between patients with and without Toxic Catecholamine Syndrome in order to identify the possible mechanism responsible for the early deaths among trauma and sepsis patients. Trauma patients with Toxic Catecholamine Syndrome were characterized as patients with a Succinic acid value above the cut-off threshold identified in Example 1. Sepsis patients with Toxic Catecholamine Syndrome were characterized as patients with a Succinic acid value above the cut-off threshold identified in Example 2.

### Results

Trauma patients with Toxic Catecholamine Syndrome had approximately 2.0 times higher levels of adrenaline and noradrenaline compared to the rest of the cohort, i.e. trauma patients without Toxic Catecholamine Syndrome (adrenaline level=496 pg/mL vs. 1004 pg/mL and noradrenaline level=846 pg/mL vs. 1671 pg/mL). Likewise, sepsis patients with Toxic Catecholamine Syndrome had approximately 2.0 times higher levels of adrenaline (1.7-fold) and noradrenaline (1.8-fold) compared to the rest of the cohort, i.e. sepsis patients without Toxic Catecholamine Syndrome (adrenaline level=227 pg/mL vs. 397 pg/mL and noradrenaline level=2720 pg/mL vs. 4905 pg/mL).

### Conclusions

Trauma and sepsis patients with Toxic Catecholamine Syndrome patients appear to have much higher levels of the catecholamine adrenaline, which we conclude is a result of sympathetic toxic hyperactivation. Of particular importance for this discovery, is that this condition can be treated by administration of pharmacological compounds that inhibit either the release of catecholamines or blockers of the adrenergic receptors inhibiting the vicious circle and normalizes the levels of circulating catecholamines.

### Example 4. Identification of a Biomarker for Early Deaths (i.e. 72-Hours) in Out-of-Hospital-Cardiac-Arrest Patients

### Method

This study included 98 adult out-of-hospital-cardiac-arrest (OHCA) patients (≥18 years) admitted to the cardiac intensive care unit. Blood samples were collected immediately upon admission to the cardiac intensive care unit. Tubes were kept at 5° C. prior to centrifugation at speed 3068, 1,800 g for 10 minutes at 5° C. at two times within one hour after obtaining the blood sample to separate plasma. Plasma was aliquoted and frozen at −80° C. for later analysis.

Gas chromatography mass spectrometry (GC-MS) was performed. The samples were pre-processed: First, samples are filtrated to remove lipids and proteins using Phree filters: An aliquot of the sample is added to the Phree filter and mixed with acetonitrile containing 1% formic acid. The filters are centrifuged 1000×g/4° C./10 mins. Filtrate from the Phree filters were dried under nitrogen flow and reconstituted in ultra-pure water prior to derivatization using MCF (methyl chloroformate). Samples were derivatized with MCF using a slightly modified version of the protocol described by Smart et al., 2010. In short: reconstituted filtrate is derivatized as described by Smart et al. and the derivatization reaction are stopped by addition of chloroform. Subsequently the chloroform is injected on the GC. All samples were analyzed in a randomized order. Analysis was performed using gas chromatography (7890B, Agilent) coupled with a quadrupole mass spectrometry detector (5977B, Agilent). The system was controlled by ChemStation (Agilent). Raw data was converted to netCDF format using Chemstation (Agilent), before the data was imported and processed in Matlab R2018b (Mathworks, Inc.) using the PARADISe software described by Johnsen et al., 2017.

Succinic acid covering percentage was 100% i.e. no missing values. Succinic acid was quantified in concentration (μmol/L).

Statistical analysis was performed using SPSS 25 (IBM Corporation, New York, NY). Receiver operating characteristic (ROC) curve analysis with the Youden index was used to determine the cut-off value of Succinic acid that maximized the sum of sensitivity and specificity as a predictor for 72-hours in-hospital mortality.

### Results

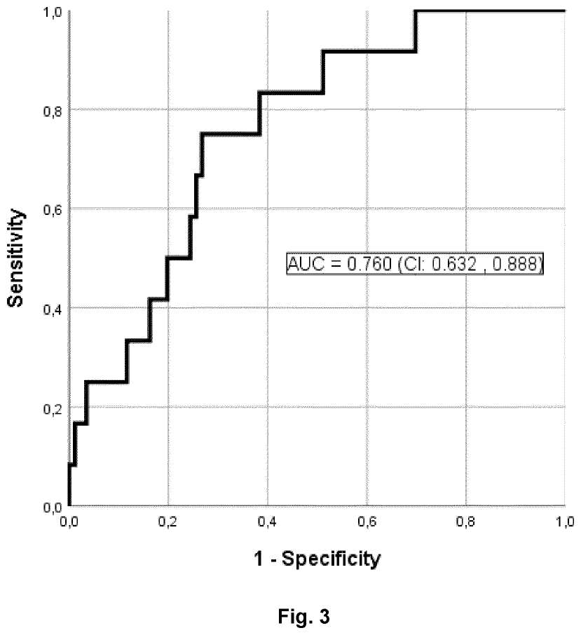

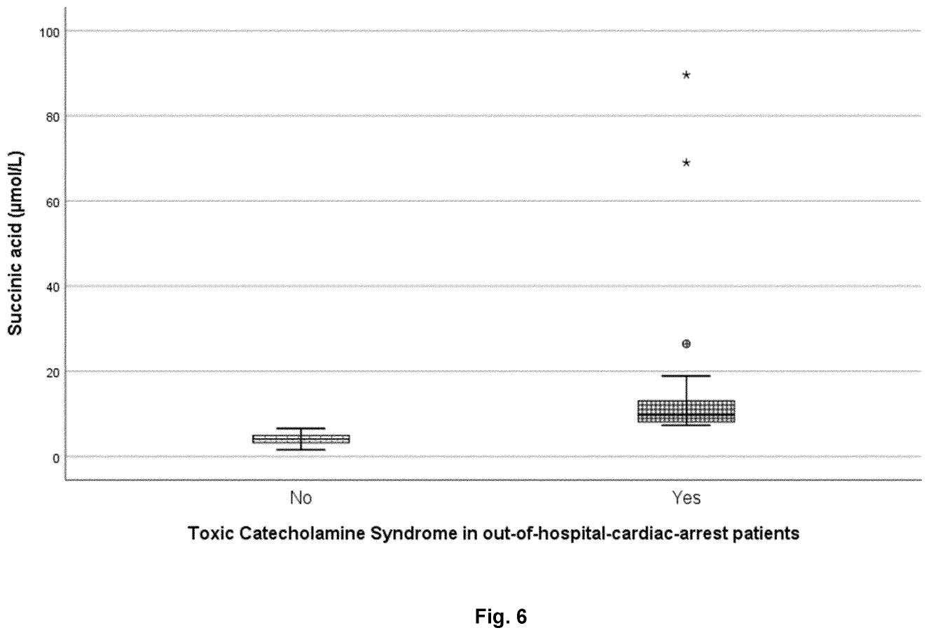

Succinic acid with a cut-off level above 7 μmol/L was able to predict the patients who die early, i.e. within 72 hours, with a sensitivity=0.750, a specificity=0.709, and a ROC AUC=0.760 (95% confidence interval 0.632, 0.888) (FIG. 3). An analysis showed that OHCA patients with Toxic Catecholamine Syndrome had approximately 3.7 times higher levels of Succinic acid in their plasma compared to OHCA patients without Toxic Catecholamine Syndrome (FIG. 6 and table 3 below).

TABLE 3
Average levels of succinic acid (μM) in plasma of OHCA patients

| | Overall | Non-TOX-OHCA | TOX-OHCA |
|---|---|---|---|
| | N = 98 | N = 64 | N = 34 |
| Succinic acid | 7.92 | 4.08 | 15.13 |
| (μM) | (3.63, 8.31) | (3.21, 4.92) | (8.11, 13.05) |

Mean (Interquartile range (IQR)) are reported.
TOX = Toxic Catecholamine Syndrome.

### Conclusions

OHCA patients suffering from Toxic Catecholamine Syndrome—identified as having a Succinic acid value above 7 μmol/L—have a significantly higher risk of early death (i.e. 72 hours). Since 50-89% of OHCA patients die in the hospital, this finding is of major importance. This biomarker therefore enables early identification of high-risk patients, for example already at admission to the hospital, and early initiation of treatment as described herein.

### Example 5. OHCA Patients with Toxic Catecholamine Syndrome Exhibit Higher Levels of Adrenaline and Noradrenaline Compared to the Rest of the Cohort

### Method

This experiment was performed on the same cohort as Example 4, and the same blood samples were used.

An Enzyme-linked immunosorbent assay (ELISA) was performed accordingly: The soluble biomarker of sympathoadrenal activation (adrenaline and noradrenaline) were measured according to the manufacturer's recommendation (2-CAT ELISA, Labor Diagnostica Nord GmbH). The covering percentage of adrenaline and noradrenaline were 100% for OHCA patients i.e. no missing values. The aim was to compare adrenaline and noradrenaline levels between patients with and without Toxic Catecholamine Syndrome in order to identify the possible mechanism responsible for the early deaths among OHCA patients. OHCA patients with Toxic Catecholamine Syndrome were characterized as patients with a Succinic acid value above the cut-off threshold identified in Example 4.

### Results

OHCA patients with Toxic Catecholamine Syndrome had approximately 2.0 times higher levels of noradrenaline (1.8-fold) and 1.4 times higher levels of adrenaline compared to the rest of the cohort, i.e. OHCA patients without Toxic Catecholamine Syndrome (adrenaline level=930 pg/mL vs. 1331 pg/mL and noradrenaline level=1274 pg/mL vs. 2352 pg/mL).

### Conclusions

OHCA patients with Toxic Catecholamine Syndrome patients have considerably higher levels of the catecholamines as a result of sympathetic toxic hyperactivation. Hence, these patients can be treated by administration of pharmacological compounds that inhibit either the release of catecholamines or blockers of the adrenergic receptors as described herein.

### REFERENCES

Abraham, J., et al., Stress cardiomyopathy after intravenous administration of catecholamines and beta-receptor agonists. J Am Coll Cardiol, 2009. 53(15): p. 1320-5
Ansari, U., et al., Clinical outcomes associated with catecholamine use in patients diagnosed with Takotsubo cardiomyopathy. BMC Cardiovasc Disord, 2018. 18(1): p. 54.
Berdowski J, Berg R A, Tijssen J G, Koster R W. Global incidences of out-of-hospital cardiac arrest and survival rates: systematic review of 67 prospective studies. Resuscitation. 2010; 81(11): 1479-87
D'Alessandro, A., et al., Plasma succinate is a predictor of mortality in critically injured patients. J Trauma Acute Care Surg, 2017. 83(3): p. 491-495
Giavarini, A., et al., Acute catecholamine cardiomyopathy in patients with pheochromocytoma or functional paraganglioma. Heart, 2013. 99(19): p. 1438-44.
Johansson, P. I., et al., Traumatic Endotheliopathy: A Prospective Observational Study of 424 Severely Injured Patients. Ann Surg, 2017. 265(3): p. 597-603.
Johnsen, L. G., et al., Gas chromatography—mass spectrometry data processing made easy. J Chromatogr A, 2017. 1503: p. 57-64.
Krug et al: World Report on Violence and Health 2002 [http://www.who.int/violence_injury_prevention/violence/world_report/en/]. Geneva: World Health Organization.
Otusanya, O., et al., A vicious cycle of acute catecholamine cardiomyopathy and circulatory collapse secondary to pheochromocytoma. Oxf Med Case Reports, 2015. 2015(10): p. 343-5.
Rakhit, S., et al., Management and Challenges of Severe Traumatic Brain Injury. Semin Respir Crit Care Med, 2021. 42(1): p. 127-144.
Rona, G., Catecholamine cardiotoxicity. J Mol Cell Cardiol, 1985. 17(4): p. 291-306.
Rudd K E, Johnson S C, Agesa K M, Shackelford K A, Tsoi D, Kievlan D R, Colombara D V, Ikuta K S, Kissoon N, Finfer S, Fleischmann-Struzek C, Machado F R, Reinhart K K, Rowan K, Seymour C W, Watson R S, West T E, Marinho F, Hay S I, Lozano R, Lopez A D, Angus D C, Murray C J L, Naghavi M. Global, regional, and national sepsis incidence and mortality, 1990-2017: analysis for the Global Burden of Disease Study Lancet. 2020 Jan. 18; 395(10219):200-211.
Smart, K. F., et al., Analytical platform for metabolome analysis of microbial cells using methyl chloroformate derivatization followed by gas chromatography-mass spectrometry. Nat Protoc, 2010. 5(10): p. 1709-29.
Wittstein, I. S., et al., Neurohumoral features of myocardial stunning due to sudden emotional stress. N Engl J Med, 2005. 352(6): p. 539-48.

1. A method of identifying a subject who is likely to benefit from treatment with a medicament which inhibits catecholamine release and/or blocks adrenergic receptors, wherein said method comprises the steps:
(a) providing a sample obtained from said subject;
(b) determining the concentration of succinic acid in said sample;
wherein a subject having an elevated succinic acid level compared to a reference level is likely to benefit from treatment with a medicament capable of inhibiting catecholamine release and/or blocking adrenergic receptors.

2. The method according to claim 1, wherein the sample is a blood sample, such as whole blood or plasma.

3. The method according to any one of claims 1-2, wherein the subject is a critically ill subject with shock, such as a trauma patient, a sepsis patient or an out-of-hospital-cardiac arrest (OHCA) patient.

4. The method according to any one of claims 1-3, wherein the elevated succinic acid level is at least 1.2 fold higher compared to a reference level, such as at least 1.3, such as at least 1.4, such as at least 1.5, such as at least 1.6, such as at least 1.7, such as at least 1.8, such as at least 1.9, such as at least 2.0, such as at least 2.5, such as at least 2.6, such as at least 2.7, such as at least 2.8, such as at least 2.9, such as at least 3.0, such as at least 3.1, such as at least 3.2, such as at least 3.3, such as at least 3.4, such as at least 3.5, such as at least 3.6, such as at least 3.7, such as at least 3.8, such as at least 3.9, such as at least 4.0 fold higher compared to a reference level.

5. The method according to any one of claims 1-4, wherein the subject is a trauma patient.

6. The method according to claim 5, wherein the reference level is obtained from a population of trauma patients, for example wherein the reference level is the average succinic acid level in a population of trauma patients.

7. The method according to any one of claims 5-6, wherein the subject likely to benefit from treatment with a medicament capable of inhibiting catecholamine release and/or blocking adrenergic receptors has a succinic acid level above a cut-off value of between 12 μmol/L and 40 μmol/L, such as between 13 μmol/L and 40 μmol/L, such as between 14 μmol/L and 40 μmol/L, such as between 15 μmol/L and 40 μmol/L.

8. The method according to any one of claims 5-7, wherein the subject likely to benefit from treatment with a medicament capable of inhibiting catecholamine release and/or blocking adrenergic receptors has a succinic acid level above about 15 μmol/L.

9. The method according to any one of claims 1-4, wherein the subject is a sepsis patient.

10. The method according to claim 9, wherein the reference level is obtained from a population of sepsis patients, for example wherein the reference level is the average succinic acid level in a population of sepsis patients.

11. The method according to any one of claims 9-10, wherein the subject likely to benefit from treatment with a medicament capable of inhibiting catecholamine release and/or blocking adrenergic receptors has a succinic acid level above a cut-off value of between 8 μmol/L and 40 μmol/L, such as between 9 μmol/L and 40 μmol/L, such as between 10 μmol/L and 40 μmol/L.

12. The method according to any one of claims 9-11, wherein the subject likely to benefit from treatment with a medicament capable of inhibiting catecholamine release and/or blocking adrenergic receptors has a succinic acid level above about 10 μmol/L.

13. The method according to any one of claims 1-4, wherein the subject is an OHCA patient.

14. The method according to claim 13, wherein the reference level is obtained from a population of OHCA patients, for example wherein the reference level is the average succinic acid level in a population of OHCA patients.

15. The method according to any one of claims 13-14, wherein the subject likely to benefit from treatment with a medicament capable of inhibiting catecholamine release and/or blocking adrenergic receptors has a succinic acid level above a cut-off value of between 5 μmol/L and 40 μmol/L, such as between 6 μmol/L and 40 μmol/L, such as between 7 μmol/L and 40 μmol/L.

16. The method according to any one of claims 13-15, wherein the subject likely to benefit from treatment with a medicament capable of inhibiting catecholamine release and/or blocking adrenergic receptors has a succinic acid level above about 7 μmol/L.

17. The method according to any one of claims 1-16, wherein the subject likely to benefit from treatment further has elevated levels of catecholamines.

18. The method according to claim 17, wherein the catecholamines are adrenaline and/or noradrenaline.

19. The method according to any one of claims 1-18, wherein the subject having elevated succinic acid levels has an increased risk of early death.

20. The method according to any one of claims 1-19, wherein the medicament which inhibits catecholamine release and/or blocks adrenergic receptors comprises or consists of a medicament selected from adrenergic receptor antagonists, catecholamine synthesis antagonist and catecholamine release inhibitors.

21. The method according to any one of claims 1-20, wherein the medicament blocks adrenergic receptors.

22. The method according to any one of claims 1-21, wherein the medicament is a beta-blocker.

23. The method according to claim 22, wherein the beta-blocker is a non-selective beta-blocker, such as bucindolol, carteolol, carvedilol, labetalol, nadolol, oxprenolol, penbutolol, pindolol, propranolol, sotalol or timolol.

24. The method according to claim 22, wherein the beta-blocker is a selective beta-blocker.

25. The method according to claim 24, wherein the selective beta-blocker is a beta-1 selective beta-blocker, such as acebutolol, atenolol, betaxolol, bisoprolol, celiprolol, esmolol, landiolol, metoprolol or nebivolol.

26. The method according to claim 24, wherein the selective beta-blocker is a beta-2 selective beta-blocker, such as butaxamine.

27. The method according to any one of claims 1-26, wherein the medicament is not an alpha-blocker.

28. The method according to any one of claims 1-27, wherein the medicament is a catecholamine synthesis antagonist.

29. The method according to claim 28, wherein the catecholamine synthesis antagonist is a tyrosine hydroxylase inhibitor, such as metyrosine.

30. The method according to any one of claims 1-29, wherein the medicament is a catecholamine release inhibitor.

31. The method according to claim 30, wherein the catecholamine release inhibitor is a natriuretic peptide, such as an atrial natriuretic peptide, an atrial natriuretic peptide homologue, such as ularitide, a ventricular natriuretic peptide, such as a recombinant ventricular natriuretic peptide, such as nesiritide.

32. The method according to any one of items 1-31, comprising administering an amount of a medicament which inhibits catecholamine release and/or blocks adrenergic receptors to said subject.

33. A medicament which inhibits catecholamine release and/or blocks adrenergic receptors for use in the treatment of a subject identified as likely to benefit from treatment with said medicament by the method as defined in any one of claims 1-32.
